(19) United States
(12) Patent Application Publication
(10) Pub. No.: US 2022/0079505 A1
(43) Pub. Date: Mar. 17, 2022

(54) Monitoring Device, System, and Method for Unrestrained Neural Signal Analysis

(71) Applicants: Electronics and Telecommunications Research Institute, Daejeon (KR); Korea Institute of Science and Technology, Seoul (KR)

(72) Inventors: Jee Hyun CHOI, Seoul (KR); Sung Q LEE, Daejeon (KR); Woo Seup YOUM, Daejeon (KR)

(21) Appl. No.: 17/485,316

(22) Filed: Sep. 24, 2021

(30) Foreign Application Priority Data
Sep. 16, 2020 (KR) 10-2020-0119280

(51) Int. Cl. A61B 5/00

(52) U.S. Cl. CPC A61B 5/4064; A61B 2503/40; A61B 5/0042

(57) ABSTRACT

The present invention relates to an unrestrained neural signal monitoring device and system configured to enable an unrestrained test by being mounted on a living test subject such as a mouse, the device comprising a neural signal detector detecting a neural signal of the test subject such as a brain wave, a first light emitting part outputting a colored light for position tracking, a second light emitting part outputting a colored light for neural signal display, and a controller for controlling the first and second light emitting parts in response to the neural signal detected through the neural signal detector. Because movement of a test subject is unrestrained, a natural activity of the test subject is enabled to thereby prevent an interactive behavior of a group from being restrained.

### CROSS REFERENCE TO RELATED APPLICATION

The present application claims priority based on Korean Patent Application No. 10-2020-0119280, filed on Sep. 16, 2020, the entire content of which is incorporated herein for all purposes by this reference.

### TECHNICAL FIELD

The present invention relates to an unrestrained neural signal monitoring device configured to enable an unrestrained experiment by being mounted on a living test subject including but not limited to a mouse, and an unrestrained neural signal analysis system configured to conduct various experiments using an unrestrained neural signal monitoring device mounted on a test subject and a method thereof.

### BACKGROUND OF THE INVENTION

What has been a problem in brains and social sciences for a long time was to understand a neural mechanism that is a basis of complex social collective behavior.

Although a neural process is required in these researches, an integrated solution for connecting a brain activity to a particular social behavior is yet to be greatly sufficient.

According to conventional researches, an individual behavior in a group was explained as an individual behavior, in the general evolutionary context, demonstrated within a group in a frame including but not limited to a collective movement (e.g., migration, defensive focus, synchronized breeding), a regional behavior (e.g., competition, hybridization, infant care) and an object number structure (e.g., hierarchy).

Although the collective behavior is not a simple aggregation of each individual behavior, a research on neural mechanism of social behavior requires a comparative measurement and analysis of individual brain level.

Although this approach emphasizes a causal relationship between a neural factor and a social behavior, most of the collective behaviors demonstrated by communication and interaction of individuals are very difficult to infer from knowledge on elements thereof.

Moreover, most of the global attributes of social group is a result of interaction adjusted among the group members, and a statistical result of a single brain research is unsatisfactory to show characteristics of social interaction.

Meantime, the brain area and neural circuit along with behavioral levels are reported as being relied on a social context in which two or more individuals are interacted. This is because most the behaviors are results of intuitive interactions among group members.

Therefore, in order to more satisfactorily explain the neurodynamic foundation on the complex social behaviors, a direct observation on brains and behaviors is required, and brain activities of several individuals must be simultaneously observable in order to research the social behaviors.

In other words, brain activities of each individual must be independently designated and simultaneously observable in a group.

At this time, because natural activities of individual objects and group's interactive behaviors are restricted when using the conventional wired devices, an unrestrained environment is required that does not restrain the natural activities of individual objects which are test subjects and group's interactive behaviors.

### DETAILED DESCRIPTION OF THE INVENTION

### Technical Subject

The present invention is devised to comply with the abovementioned necessity, and it is an object of the present invention to provide an unrestrained neural signal monitoring device enabling to simultaneously and intuitively observe a brain activity and movement without restraining the movement and brain activity of a test subject.

It is another object of the present invention to provide an unrestrained neural signal analysis system configured to conduct various experiments using an unrestrained neural signal monitoring device mounted on each test subject and a method thereof.

### Technical Solution

In one general aspect of the present invention, there may be provided an unrestrained neural signal monitoring device, the device comprising:

a power supply part to supply a driving power by being mounted on a test subject for use;

a neural signal detector detecting a neural signal of the test subject;

a first light emitting part emitting a colored light for position tracking;

a second light emitting part emitting a colored light for neural signal display; and

a controller controlling the first and second light emitting parts.

At this time, the first and second light emitting parts may output mutually different colors of lights.

Furthermore, the controller may control the second light emitting part in response to the size of the neural signal detected through the neural signal detector.

The controller may maintain the first light emitting part for a constant ON state.

The second light emitting part may be formed with a plurality of numbers, and may be respectively so formed as to output a light with a combination of various colors. The test subject may include a mouse.

The controller may periodically detect a neural signal of the test subject through the neural signal detector.

The color for position tracking may include a blue color and the color for neural signal display may include a red color.

The unrestrained neural signal monitoring device may further comprise a wireless communication part for performing a wireless communication function.

At this time, the controller may be so formed as to wirelessly communicate with other device through the wireless communication part.

The controller may wirelessly transmit the neural signal detected by the neural signal detector through the wireless communication part.

In another general aspect of the present invention, there may be provided an unrestrained neural signal analysis system, the system comprising:

one or more of the unrestrained neural signal monitoring devices;

a test box accommodated with one or more test subjects each mounted with the unrestrained neural signal monitoring device to allow the test subjects to activate therein;

a photographing part for photographing the test subjects inside the test box; and

an analyzer for grasping a moving trajectory and a neural signal generation state of each test subject by analyzing an image photographed by the photographing part.

Inner wall surfaces of the test box may be formed with a material capable of preventing reflection.

The photographing part may be disposed at an upper side of the test box to allow including a margin with a predetermined size of area toward a periphery of an image of the photographed test box.

The unrestrained neural signal analysis system may further comprise a wireless communication part for performing a wireless communication function.

At this time, the analyzer may wirelessly communicate with the unrestrained neural signal monitoring device through the wireless communication part.

The analyzer may receive neural signal information detected from the unrestrained neural signal monitoring device through the neural signal detector.

In still another general aspect of the present invention, there may be provided an unrestrained neural signal analysis method, the method comprising:

photographing an inside of a test box containing one or more test subjects each mounted with the unrestrained neural signal monitoring device and storing the inside of the test box;

grasping (identifying) a moving trajectory of each test subject by analyzing the stored image; and

grasping a neural signal generation state of each test subject by analyzing the stored image.

The unrestrained neural signal analysis method may further comprise displaying, on a display screen, the moving trajectory of each test subject and the neural signal generation state of each test subject.

### Advantageous Effects

The unrestrained neural signal monitoring device according to the present invention is formed with an unrestrained environment that does not restrain a movement of a test subject to thereby enable a natural activity of a test subject and to prevent an interactive behavior of a group from being restrained. As a result, it is possible to achieve a real time chase, identification and characterization with respect to a brain behavioral link of a freely moving test subject.

Furthermore, the restrained environment makes it possible to enlarge a research habitat and to simultaneously record a neural activity of an interacting individual.

The simultaneous monitoring of a plurality of test subjects can provide an interaction of two individuals and an opportunity of evaluating an interaction between an individual and a group as well.

The present invention configured to directly observe a brain behavior/activity from a head of a test subject can provide an intuitive framework for investigating a neural circuit of the socially-interacting and naturally-behaving test subject.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a mounted example of an unrestrained neural signal monitoring device according to the present invention,

FIG. 2 is an unrestrained neural signal analysis system according to an exemplary embodiment of the present invention,

FIG. 3 is a detailed functional structure of an unrestrained neural signal monitoring device according to an exemplary embodiment of the present invention,

FIG. 4 is an example of determination of brain wave,

FIG. 5 is an example explaining an installed position of a photographing part, and

FIG. 6 is a detailed test example according to the present invention.

### BEST MODE

The invention described hereunder may be applied with various changes and several exemplary embodiments, and particular exemplary embodiments will be described in detail through exemplary drawings and detailed descriptions.

However, it should be noted that the present invention is not limited to particular exemplary embodiments, and it will be appreciated that the present invention described is intended to embrace all such alterations, modifications, and variations that fall within the scope and novel idea of the present invention. In describing the present invention, detailed descriptions of well-known art in the art may be omitted to avoid obscuring appreciation of the invention with unnecessary details.

The terminology used herein is for the purpose of describing particular example embodiments only and is not intended to be limiting. As used herein, the singular forms may be intended to include the plural forms as well, unless the context clearly indicates otherwise.

The terms “including” or “comprising” are inclusive and therefore specify the presence of stated features, integers, steps, operations, elements, and/or components, but do not preclude the presence or addition of one or more other features, integers, steps, operations, elements, components, and/or groups thereof.

Although the terms, first, second, etc., may be used herein to distinguish various elements, these elements should not be limited by these terms. These terms may be only used to distinguish one element from another element.

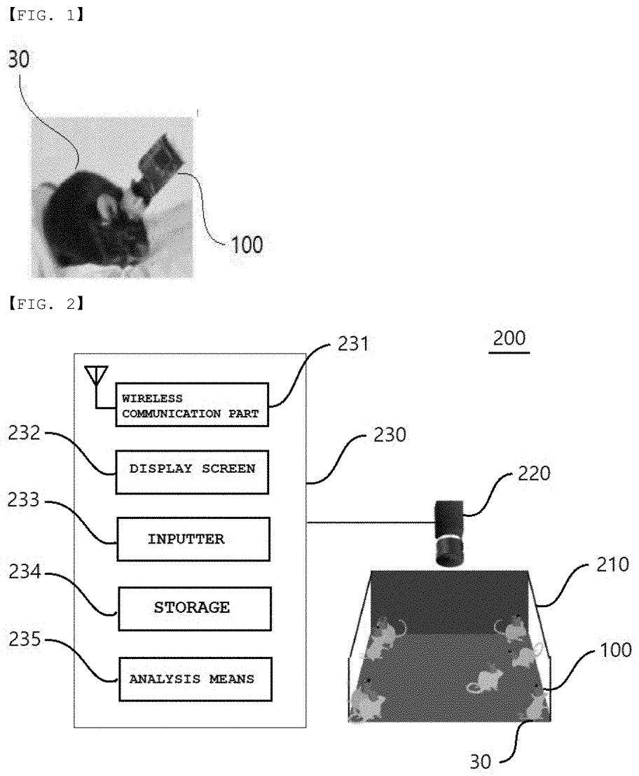

Referring to FIG. 1, an unrestrained neural signal monitoring device 100 according to the present invention may be used by being mounted on a test subject 30.

The test subject in the present invention refers to a living animal mountable with an unrestrained neural signal monitoring device 100.

Hereinafter, although the test subject is explained with a mouse as an example, the present invention is not limited thereto. That is, the test subject may be applicable to rodents such as rats, primates such as dogs and monkeys and to birds such as pigeons and eagles.

A neural signal grasped through the unrestrained neural signal monitoring device 100 may be variably available. Although the neural signal is explained hereinafter as being targeted to a short brain wave vibration signal, the present invention is not limited thereto.

Although FIG. 1 has exemplified that the unrestrained neural signal monitoring device 100 is fixedly mounted on a head portion of a mouse 30, the position and mounting method for the unrestrained neural signal monitoring device 100 to be mounted or installed on a test subject may be variably formed as necessary. However, the present invention is not particularly limited thereto.

The unrestrained neural signal monitoring device 100 may emit a colored position-chasing light for chasing of a position of a test subject 30 and a colored light for neural signal display showing that a particular neural signal has been generated from the test subject 30.

As a result, a researcher can visually ascertain a neural state of a test subject 30, and intuitively observe the neural state forming the base of the natural behavior of socially-interacting test subject.

Referring to FIG. 2, an unrestrained neural signal analysis system 200 may be disposed with an unrestrained neural signal monitoring device 100 mounted or installed on each test subject 30, a test box 210 accommodated with one or more test subjects each mounted with the unrestrained neural signal monitoring device 100 to allow the test subjects 30 to activate therein, a photographing part 220 for photographing the test subjects 30 inside the test box 210, and a main computer device 230.

The main computer device 230 may be used with an arbitrary computer device such as a desktop computer and/or a notebook computer equipped with devices for performing necessary functions, and may be interactively formed with a server system through various communication networks.

The main computer device 230 may be disposed with an analyzer 235 for overall control of unrestrained tests.

The analyzer 235 may be realized by using a computer program executed by being installed on the main computer device 230 and may grasp a moving trajectory and a neural signal generation state of each test subject 30 by analyzing an image photographed by the photographing part 220.

The analyzer 235 may be variably formed as necessary.

For example, the analyzer 235 may perform various functions including but not limited to an overall control of unrestrained tests, test data analysis, and process and output of test results while interfacing with a researcher through a GUI (Graphic User Interface) environment.

As a result, the researcher can intuitively observe the movement of test subject 30 realized within the test box 210 and neural signal generation state and simultaneously ascertain various test results through automated analyses.

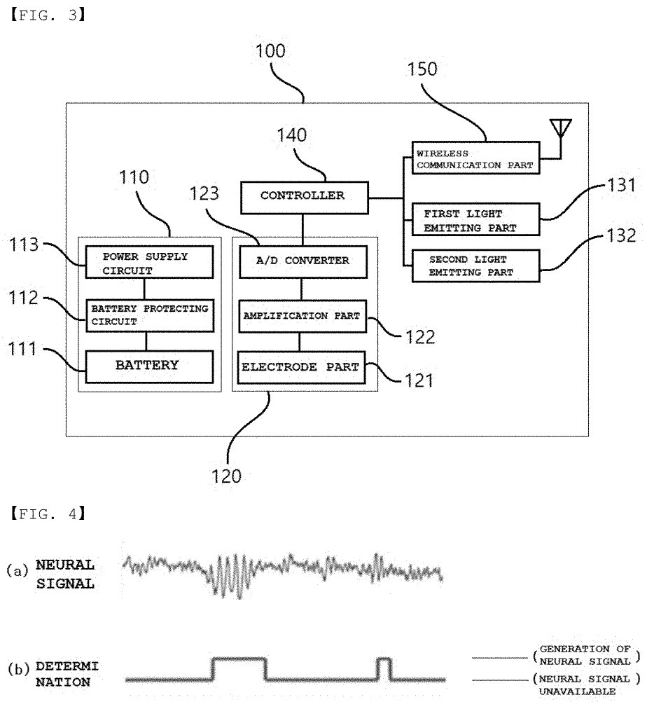

FIG. 3 illustrates a detailed functional structure of an unrestrained neural signal monitoring device 100 according to an exemplary embodiment of the present invention, and may be formed by including a power supply part 110, a neural signal detector 120 detecting a neural signal of a test subject, a first light emitting part 131 outputting a colored light for position chasing/tracking, a second light emitting part 132 outputting a colored light for neural signal display, and a controller 140 controlling the first and second light emitting parts 131, 132. The number of first and second light emitting parts 131, 132 may be variably formed as necessary.

The power supply part 110 may supply a power source necessary for operation of the unrestrained neural signal monitoring device 100 and may be variably formed.

As one of examples, the power supply part 110 may be rechargeably formed by including a rechargeable battery 111, a battery protecting circuit 112, and a power supply circuit 113 for outputting a DC voltage of a particular level, and the present invention is not however limited thereto.

The neural signal detector 120 may detect a neural signal of a test subject.

FIG. 3 illustrates an exemplary embodiment of a neural signal detector 120 formed by including an electrode part 121 inserted into a brain, an amplification part 122 amplifying an electric signal detected through the electrode part 121, and an A/D (Analog to Digital) converter 123 converting a signal amplified through the amplification part 122 to a digital signal, in order to detect a short vibration signal generated from the brain.

However, the neural signal detector 120 may be variably formed as necessary and therefore, the present invention is not limited thereto.

In order to accurately detect a neural signal of a test subject, the electrode part 121 must be accurately disposed on a portion where a neural signal is generated.

The first and second light emitting parts 131, 132 may respectively output a colored light for position chasing/tracking and a colored light for neural signal display.

The first and second light emitting parts 131, 132 may be formed by using an LED (Light Emitting Diode). However, the present invention is not limited thereto.

Because the position chasing color and the neural signal display color must be distinguished, the first and second light emitting parts 131, 132 respectively output mutually different colors of lights.

Because the light outputted by the first light emitting part 131 is used for position chasing of a test subject, the first light emitting part 131 can maintain a constant ON state while tests are being performed.

Because the second light emitting part 132 outputs a colored light for neural signal display that shows whether a neural signal has been generated from a test subject, the second light emitting part may be turned ON depending on whether a neural signal has been generated.

Two or more mutually different neural signals may be detected from one test subject. For example, tests may be performed while an alpha wave of brain is also detected. Toward this end, two or more second light emitting parts 132 may be included.

When the second light emitting part 132 is formed with a plural number, a light combination of mutually different colors may be outputted in order to divisibly display the presence or absence of generation of mutually different neural signals.

Hereinafter, the color for position chasing outputted from the first light emitting part 131 will be explained as a blue color, and the color for neural signal display outputted from the second light emitting part 132 in response to the generation of neural signal will be explained as a red color.

Therefore, although there may be no separate explanation, the blue color will mean the color for position chasing, and the red color will mean the color for neural signal display. The abovementioned explanation is intended for convenience sake and is not intended to limit the position chasing color and the neural signal display color.

The controller 140 may maintain the first light emitting part 131 at a constant ON state, and may control ON/OFF states of the second light emitting part 132 in response to a detected state of neural signal.

The controller 140 may detect a neural signal using various methods through the neural signal detector 120. For example, the controller 140 may be so formed as to periodically detect a neural signal of a test subject through the neural signal detector 120 at a predetermined time interval (e.g., 10 ms). Furthermore, the controller 140 may control the ON/OFF states of the second light emitting part 132 in response to the size of detected neural signal.

FIG. 4 is an example of determination in response to a short vibration signal generated from a brain, where the controller 140 determines that a neural signal has been generated when the size of the detected neural signal is greater than a reference level to thereby control the second light emitting part 132 at an ON state, and determines that a neural signal has not been generated when the size of detected neural signal is less than a reference level to thereby control the second light emitting part 132 at an OFF state.

As explained above, when the neural signal detector 120 is formed by including the A/D converter 123, the controller 140 may be formed by using a microprocessor that can process a digital value generated from the A/D converter 123.

The unrestrained neural signal monitoring device 100 may be formed by including a wireless communication part 150 that performs a wireless communication function.

The wireless communication part 150 may be so configured as to communicate with other device including but not limited to a main computer device 230 using an arbitrary wireless communication method such as Bluetooth and the like. At this time, the controller 140 of the unrestrained neural signal monitoring device 100 may communicate with the analyzer 235 of the main computer device 230 through the wireless communication part 150 to thereby exchange the necessary information.

For example, the controller 140 may wirelessly transmit the neural signal detected by the neural signal detector 120 through the wireless communication part 150, and control the unrestrained neural signal monitoring device 100 in response to a control signal received from the wireless communication part 150.

Referring to FIG. 2 again, the main computer device 230 may be disposed with a wireless communication part 231 that can communicate using various wireless communication methods including Bluetooth, a display screen 232, an inputter (input device, 233) including a keyboard, a mouse and a touch panel, and storage 234 storing various types of information. Particularly, the main computer device 230 may be installed with analyzer 235 that performs an overall control of the unrestrained tests.

The test box 210 may be accommodated with one or more living test subjects 30 mounted with the unrestrained neural signal monitoring device 100.

The test box 210 may be variably formed. Although the given drawings have illustrated an example of a square box shape, the shape, size, structure and material of the test box 210 may be variably formed as necessary. The present invention is not particularly limited thereto.

The photographing part 220 may photograph an interior of the test box 210 that activates by being accommodated with the test subject 30.

The photographing part 220 may be formed using a camera device equipped with necessary performances (e.g., resolution, the number of frames per second, colors, etc.) and capable of photographing a video.

As a detailed example, the photographing part 220 may be formed using a streaming spectrum video recorder. The present invention is not limited thereto and may be variably formed as necessary.

Because movement of test subject is grasped through an image photographed by the photographing part 220, the photographing part 220 must be so disposed as to well photograph an entire area of the interior of the test box 210.

Furthermore, photographing environments including but not limited to an illumination (lighting) must be adequately adjusted to allow the colored light for position chasing outputted by the first light emitting part 131 and the colored light for neural signal display outputted by the second light emitting part 132 to be accurately and clearly photographed.

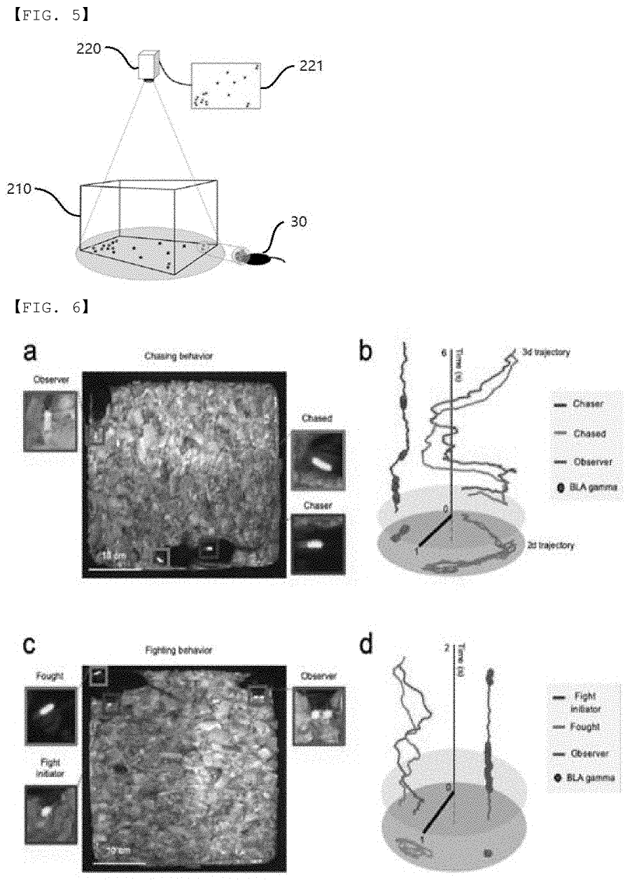

Referring to FIG. 5, the photographing part 220 may be disposed at an upper side from the test box 210 that is discrete as much as a predetermined distance.

Each frame 221 may be displayed with the photographed colored light for position chasing and colored light for neural signal display outputted from respective unrestrained neural signal monitoring devices 100. An installed height of the photographing part 220 may be so formed as to include a sufficient area of margin toward a periphery of the photographed frame image in order to improve the accuracy and efficiency of analysis.

The photographing part 220 must be adequately adjusted in its exposed time. If the camera exposure time is too short, there may be generated an omission error, and if the camera exposure time is too long, adjacent pixels may be merged due to light saturation.

The analyzer 235 may exchange various types of information related to tests by wirelessly communicating with the unrestrained neural signal monitoring device 100 through the wireless communication part 231.

As a detailed example, the analyzer 235 may receive the neural signal information detected by the controller 140 of the unrestrained neural signal monitoring device 100 through the neural signal detector 120 and store the information in the storage 234. The neural signal information (raw data) detected by the controller 140 of the unrestrained neural signal monitoring device 100 through the neural signal detector 120 may be used for various purposes including but not limited to background brain wave analysis.

Furthermore, the analyzer 235 may transmit information related to the tests to the unrestrained neural signal monitoring device 100. For example, the analyzer 235 may transmit a reference value that determines presence or absence of generation of neural signal to the unrestrained neural signal monitoring device 100.

That is, the neural signal may include conventionally-generated noises detected by the neural signal detector 120, and these noises may demonstrate intrinsic physical properties in response to a test subject. Under these circumstances, the analyzer 235 may transmit a neural signal determinative reference value experimentally determined relative to each test subject to the unrestrained neural signal monitoring device 100 mounted on each test subject.

Then, the controller 140 on each unrestrained neural signal monitoring device 100 may determine that the neural signal has been generated when the neural signal detected by the neural signal detector 120 is greater than the received determinative reference value to thereby turn ON the second light emitting part 132.

Thus, the determination of whether or not there has been generated a neural signal on all test subjects can be realized under the same condition through the hitherto mentioned processes.

Now, an unrestrained neural signal analysis method according to an exemplary embodiment of the present invention will be described.

First, while the tests are being made, the analyzer 235 may photograph an inside of the test box 210 containing one or more test subjects mounted with the unrestrained neural signal monitoring device 100 and store the same in the storage 234.

Furthermore, the analyzer 235 may grasp a moving trajectory of each test subject by analyzing the stored image.

The automatic chase of the position of test subject is important in quantitatively analyzing group behaviors and social interactions.

Furthermore, the analyzer 235 may grasp a neural signal generation state of each test subject by analyzing the stored image.

FIG. 6 illustrates one test example performed in accordance with the present invention, where a border related brain state of static mouse under a fierce situation among the mice is shown.

FIG. 6a illustrates a momentary chasing/being chased scene, where a brain vibration was not observed in the chaser mouse and the chased mouse, but observed in the third static mouse (observer).

FIG. 6b illustrates an example of a 6-second trajectory of three mice and generation of brain behaviors (activities) (red dot) in FIG. 6a that are shown by using a disc coordinate.

FIG. 6c illustrates a fighting momentary scene, where the brain vibration was not observed in a mouse participated in the fighting but observed in the third static mouse (observer).

FIG. 6d illustrates an example of a 2-second trajectory of three mice and generation of brain behaviors (activities) in FIG. 6c that are shown by using a disc coordinate.

As shown in FIGS. 6a to 6d, it can be easily seen that all trajectories are prone to be near the walls, and it can be seen that the brain vitalization is demonstrated in a mouse that takes precautions against an aggressive behaviors of other mice.

In other words, although the static mouse as an observer is not actually participated in the fierce situation, the brain vibrations were greatly generated from the observer mouse when one mouse aggressively chases or picks a fight against the other mouse.

As shown from these examples, effects of dynamic social interactions on brain behaviors (activities) of many test subjects were able to be observed by simultaneously quantifying the same.

Meantime, the unrestrained neural signal analysis method may be embodied on a computer readable recording medium using a computer readable code.

The computer readable recording medium may include all kinds of recording devices stored with data readable by a computer system.

Although the abovementioned embodiments according to the pre sent invention have been described and illustrated in detail with reference to the above specific examples, the embodiments are, however, intended to be illustrative only, and thereby do not limit the scope of protection of the present invention. Thereby, it should be appreciated by the skilled in the art that changes, modifications and amendments to the above examples may be made without deviating from the scope of protection of the invention.

### DESCRIPTION OF REFERENCE NUMERALS

30: test subject
100: unrestrained neural signal monitoring device
110: power supply part
111: battery
112: battery protecting circuit
113: power supply circuit
120: neural signal detector
121: electrode part
122: amplification part
123: A/D converter
131: first light emitting part
132: second light emitting part
140: controller
150: wireless communication part
200: unrestrained neural signal analysis system
210: test box
220: photographing part
230: main computer device
231: wireless communication part
232: display screen
233: inputter (input device)
234: storage
235: analyzer

1. An unrestrained neural signal monitoring device, the device comprising:
a power supply part to supply a driving power by being mounted on a test subject for use;
a neural signal detector detecting a neural signal of the test subject;
a first light emitting part outputting a colored light for position tracking;
a second light emitting part outputting a colored light for neural signal display; and
a controller controlling the first and second light emitting parts,
wherein the first and second light emitting parts output mutually different colors of lights, and
the controller controls the second light emitting part in response to the size of the neural signal detected through the neural signal detector.

2. The device of claim 1, wherein the controller maintains the first light emitting part for a constant ON state.

3. The device of claim 1, wherein the second light emitting part is formed with a plurality of numbers, and each part outputs lights of mutually different colors.

4. The device of claim 1, wherein the test subject includes a mouse.

5. The device of claim 1, wherein the controller periodically detects a neural signal of the test subject through the neural signal detector.

6. The device of claim 1, wherein the color for position tracking includes a blue color and the color for neural signal display includes a red color.

7. The device of claim 1, further comprising a wireless communication part for performing a wireless communication function, wherein the controller is so formed as to wirelessly communicate with other device through the wireless communication part.

8. The device of claim 1, wherein the controller wirelessly transmits the neural signal detected by the neural signal detector through the wireless communication part.

9. An unrestrained neural signal analysis system, the system comprising:
one or more of the unrestrained neural signal monitoring devices of claim 1;
a test box accommodated with one or more test subjects each mounted with the unrestrained neural signal monitoring device to allow the test subjects to activate therein;
a photographing part for photographing the test subjects inside the test box; and
an analyzer for grasping a moving trajectory and a neural signal generation state of each test subject by analyzing an image photographed by the photographing part.

10. The system of claim 9, wherein an inner wall surfaces of the test box is formed with a material capable of preventing reflection.

11. The system of claim 9, wherein the photographing part is disposed at an upper side of the test box to allow including a margin with a predetermined size of area toward a periphery of an image of the photographed test box.

12. The system of claim 9, further comprising a wireless communication part for performing a wireless communication function, wherein the analyzer wirelessly communicates with the unrestrained neural signal monitoring device through the wireless communication part.

13. The system of claim 9, wherein the analyzer receives neural signal information detected from the unrestrained neural signal monitoring device through the neural signal detector.

14. An unrestrained neural signal analysis method, the method comprising:
photographing an inside of a test box containing one or more test subjects each mounted with the unrestrained neural signal monitoring device of claim 1 and storing the inside of the test box;
grasping a moving trajectory of each test subject by analyzing the stored image; and
grasping a neural signal generation state of each test subject by analyzing the stored image.

15. The method of claim 14, further comprising displaying, on a display screen, the moving trajectory of each test subject and the neural signal generation state of each test subject.

16. A computer system readable recording medium recorded with a computer program for implementing the unrestrained neural signal analysis method of claim 14 in a computer system.

17. A computer system readable recording medium recorded with a computer program for implementing the unrestrained neural signal analysis method of claim 15 in a computer system.
